(19) United States
(12) Patent Application Publication
(10) Pub. No.: US 2010/0068772 A1
(43) Pub. Date: Mar. 18, 2010

(54) Solubilization of algae and algal materials

(76) Inventor: Robert Downey, Centennial, CO (US)

Correspondence Address: Alan J. Grant, Esq.;c/o Carella, Byrne, Bain,, Gilfillan, Cecchi, Stewart & Olstein, 5 Becker Farm Road, Roseland, NJ, 07068, US

(21) Appl. No.: 12/584,300

(22) Filed: Sep. 3, 2009

Related U.S. Application Data
(60) Provisional application No. 61/190,932, filed on Sep. 4, 2008.

(51) Int. Cl. C12P 5/02; C10L 1/00; C12P 1/04; C12P 7/64

(52) U.S. Cl. 435/134; 44/307; 435/170; 435/166

(57) ABSTRACT

Methods for solubilizing algae or algal material are provided to facilitate the recovery of oil or lipids, as well as hydrocarbons and carbohydrates, from algae or algal material. The methods involve contacting algae or algal material with an oxoacid ester or thioacid ester of phosphorus or a mixture of an oxoacid of phosphorus and/or an alcohol to form a mixture thereof under conditions effective to solubilize the algae or algal material. These methods optionally further comprise bioconversion of the solubilized algae or algal material to form a composition suitable for recovery of oils and non-oil chemicals.

This application claims priority of U.S. Provisional Application 61/190,932, filed 4 Sep. 2008, the disclosure of which is incorporated herein by reference in its entirety.

### FIELD OF THE INVENTION

The present invention relates to the field of solubilization and recovery of materials from plant matter, especially algae and algal materials.

### BACKGROUND OF THE INVENTION

Increased demand for crude oil and petroleum-based products, competing demands between foods and other biofuel sources and increased demand for food worldwide has strongly increased research and development in algaculture, or algae farming, for the production of vegetable oil, biodiesel, bioethanol, biogasoline, biomethanol, biobutanol and other biofuels.

The United States Department of Energy estimates that if algae fuel replaced all the petroleum fuel in the United States, it would require 15,000 square miles (38,849 square kilometers), which is a few thousand square miles larger than the state of Maryland. This is less than 1/7th the area of corn harvested in the United States in 2000.

Microalgae have much faster growth rates than terrestrial crops. The per unit area yield of oil from algae is estimated to be from between 5,000 to 20,000 gallons per acre, per year (4.6 to 18.4 l/m² per year); this is 7 to 30 times greater than the next best crop, Chinese tallow (699 gallons). Algae can also grow on marginal lands, such as in desert areas where the groundwater is saline.

The difficulties in efficient biodiesel production from algae lie in finding an algal strain with a high lipid content and fast growth rate that isn't too difficult to harvest, a cost-effective cultivation system (i.e., type of photobioreactor or other cultivation system) that is best suited to that strain, and efficient methods of extracting oils from the algae, along with other materials that may also be converted to useful fuels or products.

Research into algae for the mass-production of oil is mainly focused on microalgae; organisms capable of photosynthesis that are less than 2 mm in diameter, including the diatoms and cyanobacteria; as opposed to macroalgae, e.g. seaweed. This preference towards microalgae is due largely to its less complex structure, fast growth rate, and the high oil content of some species.

Algae are a large and diverse group of simple, typically autotrophic organisms, ranging from unicellular to multicellular forms. The largest and most complex marine forms are called seaweeds. Most are photosynthetic, like plants, and “simple” because they lack many of the distinct organs found in land plants. Though the prokaryotic cyanobacteria (commonly referred to as blue-green algae) were traditionally included as “algae” in older textbooks, many modern sources regard this as outdated and restrict the term algae to eukaryotic organisms. All true algae therefore have a nucleus enclosed within a membrane and chloroplasts bound in one or more membranes.

Algae lack the various structures that characterize land plants, such as phyllids and rhizoids in nonvascular plants, or leaves, roots, and other organs that are found in tracheophytes. They are distinguished from protozoa in that they are photosynthetic. Many are photoautotrophic, although some groups contain members that are mixotrophic, deriving energy both from photosynthesis and uptake of organic carbon either by osmotrophy, myzotrophy, or phagotrophy. Some unicellular species rely entirely on external energy sources and have reduced or lost their photosynthetic apparatus. Some algae can also grow in the absence of light using, for example, glucose as sole carbon source or can be genetically modified to grow on sugar as the sole carbon source without light (see, for example, U.S. Patent Publication 20070191303).

All algae have photosynthetic machinery ultimately derived from the cyanobacteria, and so produce oxygen as a byproduct of photosynthesis, unlike other photosynthetic bacteria such as purple and green sulfur bacteria.

There are three well-known methods to extract oil from algae: The expeller/press, which involves the use of a mechanical press to extract the oil; hexane solvent oil extraction, which can be used in isolation or in combination with an expeller/press, and whereby the oil dissolves in the cyclohexane and is then recovered via distillation; and supercritical fluid extraction, where CO₂ is liquefied under pressure and heated to the point that it has the properties of both a liquid and gas. This liquefied fluid then acts as the solvent in extracting the oil. Other oil extraction methods include enzymatic extraction, which uses enzymes to degrade the cell walls with water acting as the solvent; osmotic shock, which involves a sudden reduction in osmotic pressure, causing cells in solution to rupture; and ultrasonic assisted extraction, whereby ultrasonic waves are used to create cavitation bubbles in a solvent material, when these bubbles collapse near the cell walls, it creates shock waves and liquid jets that cause those cells walls to break and release their contents into the solvent.

The amount of oil that may be recovered by these various methods varies, and in general the highest recoveries result from the most expensive processes. In all of these methods, the non-oil portions of the algae are either discarded or utilized in one or more low-value applications.

### BRIEF SUMMARY OF THE INVENTION

In accordance with one aspect of the present invention, algae or algal material is treated with a liquid that contains at least one oxyacid ester of phosphorus and/or at least one thio acid ester of phosphorus. The treating is effected under conditions that liquefy (solubilize) the algae or algal material. In one preferred embodiment, the liquid includes water.

As referred to herein, a “liquid that contains at least one” of an oxoacid ester of phosphorus or a thioacid ester of phosphorus may be produced in solution from the appropriate oxoacid or thioacid and the appropriate alcohol. Where referred to throughout this disclosure a “liquid that contains at least one” of an oxoacid ester of phosphorus or a thioacid ester of phosphorus shall mean either “a liquid containing at least one of an oxoacid ester of phosphorus or a thioacid ester of phosphorus” and/or a liquid comprising the appropriate oxoacid or thioacid and the appropriate alcohol.

As used herein, “solubilize” means that at least a portion of the algae or algal material is liquefied. All, a portion or none of the “solubilized” or “liquefied” algae or algal material may be soluble in the treating liquid. The treating in accordance with the invention is employed to obtain from the algae or algal material the oil portion thereof as well as all or a portion of the non-oil portion thereof. The non-oil portions of the algae generally include cellulosic and hemicellulosic material, polysaccharides, heterosaccharides, carbohydrates, proteins and fatty acids. Recovery of all or a portion of these materials would thus increase the overall conversion of algae to useful products, and provide access to them by cellulases and fermentation enzymes, and/or direct conversion to methane by methanogenic or other microbiological consortia. This solvent treatment process enables oil recoveries, and conversion of the non-oil components to higher-value chemicals and materials.

Thus, products recovered by said treating may include the oils, lipids, hydrocarbons and carbohydrates from the solubilized or liquefied algae.

In another aspect, the present invention is directed toward a composition comprising solubilized organophosphorus ester derivatives of algae or algal material.

A further aspect of the invention is directed toward a bioconversion method that includes contacting a composition described herein with a bioconversion agent under suitable conditions, wherein said composition is formed by solubilizing algae or algal material with the said at least one oxyacid ester of phosphorus and/or at least one thio acid ester of phosphorus.

In one embodiment, the organophosphorus system is added to or combined in a process containing algae or algal materials to rapidly solubilize (liquefy) the algae or algal materials.

In another embodiment, the process for solubilizing algae and/or algal material is enhanced by sonication (i.e., the application of sonic waves).

In a further embodiment, the process for solubilizing algae and/or algal material is improved by increase in temperature (i.e., solubilization rates increase with increased temperature) or by a change in pH.

In a still further embodiment, the process of solubilizing algae and/or algal material is further improved by foaming or misting of the composition with a gas.

In an additional aspect, the present invention relates to a method of solubilizing or liquefying algae or algal material using (i) an oxoacid ester and/or thioacid ester of phosphorus or (ii) a mixture of an oxoacid or thioacid of phosphorus and an alcohol.

### DETAILED DESCRIPTION OF THE INVENTION

One aspect of the present invention is directed toward a method of solubilizing algae or algal material. The method includes providing algae or algal material and providing an oxoacid ester or thioacid ester of phosphorus or a mixture of an oxoacid or thioacid of phosphorus and an alcohol. A mixture of the algae or algal material and the oxoacid ester or thioacid ester of phosphorus or the mixture of the oxoacid or thioacid of phosphorus and alcohol is formed. The mixture is then treated under conditions effective to partially or completely solubilize the algae or algal material. The products resulting from such treatment may include lipids, oils, carbohydrates, proteins, fatty acids, hydrogen, carbon dioxide and other chemicals.

In general, the algae or algal material may be macroalgae, microalgae, diatoms or cyanobacteria and the treating step may be carried out at a temperature of 20 to 150° C., preferably at a temperature of 80 to 100° C. at a pH range of 1 to 9.

In certain embodiments, an oxoacid ester or thioacid ester of phosphorus is provided. In other embodiments, a mixture of an oxoacid or thioacid of phosphorus an the alcohol is provided. The algae or algal material may be fully solubilized or may be partially solubilized as a result of the treating and provide one or more of hydrocarbons, carbohydrates, lipids or oils from the algae, a valuable process improvement in the production of oil or polysaccharides from algae.

It is well known that hydrolysis equilibria are reversible for many chemicals. Phosphite esters are no exceptions (see Scheme 1 for an example). As shown, the production of the ester can proceed from left to right in each equilibrium step starting with P(OEt)₃ and water, or from right to left starting from phosphorous acid and ethanol at the lower right of the Scheme. Starting with 3 equivalents of EtOH and an equivalent of phosphorous acid and then removing the water (e.g., with molecular sieves) produces mainly P(OEt)₃.

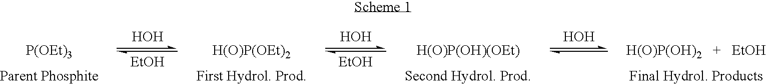

It is possible to start with phosphorous acid and the required alcohol to make a mixture of the first hydrolysis product and the second hydrolysis product for use as the active pretreatment medium or to start with the first hydrolysis product, and by adding the correct amount of water, make the same mixture as starting with phosphorous acid and the required alcohol.

It is generally possible to proceed in either direction of an equilibrium or sequence of equilibria. This process is governed by Le Chatelier's Principle.

The alcohols (see Table 1, below) from which A, (ethanol), B (ethylene glycol), C (propylene glycol), and D (2,2-dimethylpropylene-1,3-diol) are made are commercially inexpensive, are manufactured in large volumes, and are of very considerable industrial importance.

TABLE 1

| | | |
|---|---|---|
| H(O)P(OEt)₂ A | 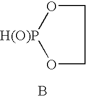 | 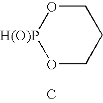 |
| 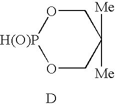 | EtOP(OEt)₂ E | 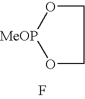 |
| 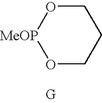 | 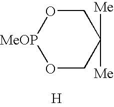 | H(O)POH(OEt) I |
| 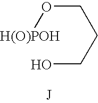 | 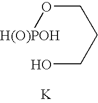 | 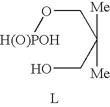 |

In Schemes 2, 3, and 4 (below), the polyols from which N, R, and V in these schemes are made are glycerol, trimethylol propane, and pentaerythritol, respectively (see Table 1, above). These polyols are very cheap and are made in large volumes (i.e., glycerol is an overly abundant byproduct of the biodiesel industry, trimethylol propane is used in polyurethane manufacture, and pentaerythritol is made in over 100 million pound quantities per year, most of which is used in alkyd resins and lubricants). Although the parent bicyclic phosphite M in Scheme 2 is known, it would not form in the proposed reaction of glycerol and phosphorous acid, because of its strained bonds and the fact that its formation would require the presence of a catalyst. A catalyst is also required for the analogous formations of the toxic parent phosphite Q in Scheme 3 and the non-toxic parent phosphite U shown in Scheme 4. It should be noted that neither first nor second hydrolysis products for the phosphite esters in Schemes 2-4 are commercially available, nor are there reports of their isolation to date.

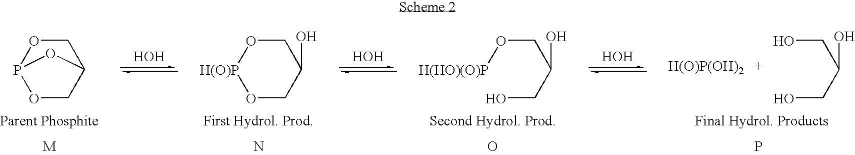

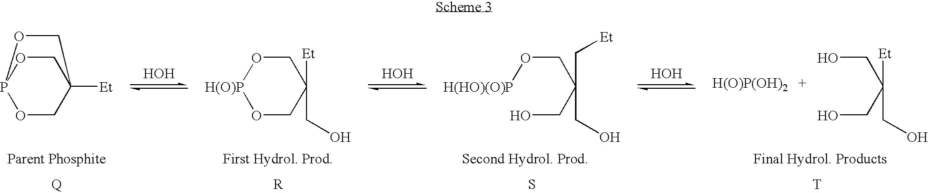

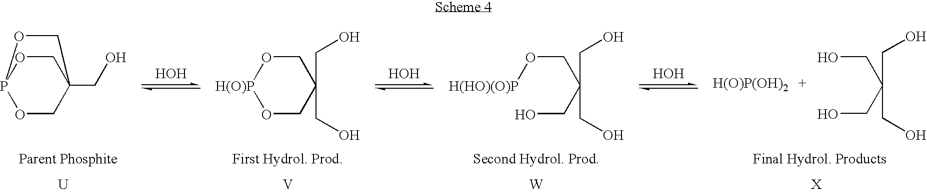

Synthesis of parent phosphite esters for subsequent hydrolysis (to make the desired ratio of first to second hydrolysis products) requires expense, time, and energy, which can be avoided by starting with phosphorous acid and the desired alcohol, diol, triol, or tetraol, followed by removing the appropriate amount of water. The mixture of active agents is created by proceeding from the final hydrolysis products and working toward parent phosphites but not actually synthesizing them.

The first hydrolysis products A-D of the parent phosphites E-H, respectively, are effective agents for coal. Compounds A, B, and D are commercially available, but C can be synthesized. It should be noted that A-D by themselves are also effective in the presence of some water to make a mixture of first and second hydrolysis products I-L.

One skilled in the art would recognize that thiophosphoryl compounds, those bearing the P═S functionality, may be substituted for related phosphoryl derivatives. Such substitution of a sulfur for one or more oxygens in a phosphorous oxoacid, an oxoacid ester, a phosphoric oxoacid, or a phosphoric acid ester is possible because thiophosphorous and thiophosphoric compounds are well known. However, such sulfur containing compounds could be more expensive and pose environmental problems.

As referred to herein, a “liquid that contains at least one” of an oxoacid ester of phosphorus or a thioacid ester of phosphorus may be produced in solution from the appropriate oxoacid or thioacid and the appropriate alcohol. Where referred to throughout this disclosure a “liquid that contains at least one” of an oxoacid ester of phosphorus or a thioacid ester of phosphorus shall mean either “a liquid containing at least one of an oxoacid ester of phosphorus or a thioacid ester of phosphorus” and/or a liquid comprising the appropriate oxoacid or thioacid and the appropriate alcohol.

The algae or algal material suitable for use in the invention includes, but is not limited to, macroalgae, microalgae, diatoms or cyanobacteria. In one embodiment, the treating step may be carried out at a temperature of 20 to 150° C., preferably at a temperature of 80 to 100° C. In another embodiment, the treating step is carried out at a pH range of 1 to 9. In one example of the invention, a oxoacid ester or thioacid ester of phosphorus is an ester of phosphorous acid, phosphoric acid, hypophosphorous acid, polyphosphoric acid, or mixtures thereof. In another example, the oxoacid of phosphorus is selected from phosphorous acid, phosphoric acid, hypophosphorous acid, polyphosphoric acid, or mixtures thereof. A thioacid ester of phosphorus would be selected from thiophosphorous and thiophosphoric acids.

Suitable alcohols for use in the methods of the invention include methanol, ethanol, ethylene glycol, propylene glycol, glycerol, pentaerythritol, trimethylol ethane, trimethylol propane, trimethylol alkane, alkanol, polyol, or mixtures thereof. Mixtures used in the invention preferably have a ratio of the oxoacid of phosphorus to the alcohol of from 10:1 to 1:10.

The methods of the present invention include regulating the water content of the mixture before or during treating and such regulation may be carried out by removing water. Suitable techniques for doing so include molecular sieving, distillation, or adding a dehydrating agent to the mixture.

Another aspect of the present invention is directed toward a composition comprising solubilized organophosphorous ester derivatives of algae or algal material.

The methods of the preset invention optionally include regulating the water content of the mixture before or during treating, via foaming (where liquid is the continuous phase and gas is the discontinuous phase) or misting (where liquid is the discontinuous phase and gas is the continuous phase) of the mixture or mixture constituents with a gas or gases.

The methods of the present invention also optionally include sonicating the mixture during or after the treating.

The methods of the present invention also optionally include adding a bioconversion agent to the mixture after treating with said oxoacid or thioacid esters of phosphorus and/or to one or more of the products recovered from the mixture. Suitable bioconversion agents include methanogens, a variety of facultative anaerobes, acetogens, and other species capable of converting some of the solubilized algal materials to hydrocarbons, fatty acids, carbohydrates and other useful chemicals.

In accordance with the foregoing, a further aspect of the present invention is directed toward a bioconversion method. This method includes providing the treated algae or algal composition (as described hereinabove), or one or more of the products recovered therefrom, with a bioconversion agent under conditions effective to bioconvert one or more of the products resulting from the treatment. Useful bioconversion agents include methanogens, a variety of facultative anaerobes, acetogens, and other microbial species. Suitable bioconversion includes formation of methane, hydrocarbons, fatty acids, carbohydrates and other useful chemicals and gases.

Thus, the methods of the invention are useful in treating algae or algal materials to render the products obtained from such treatment suitable, for example, for further processing in bioconversion, including formation of methane.

As used herein, the terms “algae” or “algal material” broadly encompass a large and diverse group of simple, typically autotrophic eukaryotic organisms, ranging from unicellular to multicellular forms. The largest and most complex marine forms, or macroalgae, are often called seaweeds. Forms of microalgae, organisms capable of photosynthesis that are less than 2 mm in diameter, include cyanobacteria and diatoms. Most algae are photosynthetic, like plants, and “simple” because they lack many of the distinct organs found in land plants. Algae or algal, as used herein, describes compounds comprising or related to algae in its various forms.

Algae useful in practicing the methods of the invention include, but are not limited to, those of the genus Dunaliella, Chlorella, Nannochloropsis, or Spirulina. In one embodiment, such algae include Dunaliella Bardawil, Dunaliella salina, Dunaliella primolecta, Chlorella vulgaris, Chlorella emorsonii, Chlorella minutissima, Chlorella sorokiniana, Chlorella vulgaris, Spirulina platensis, Cyclotella cryptica, Tetraselmis suecica, Monoraphidium, Botryococcus braunii, Stichococcus, Haematococcus pluvialis, Phaeodactylum tricornutum, Tetraselmis suecica, lsochrysis galbana, Nannochloropsis, Nitzschia closterium, Phaeodactylum tricornutum, Chlamydomas perigranulata, Synechocystisf, Tagetes erecta or Tagetes patula.

The methods of the present invention can also be applied to materials derived from genetically modified organisms, such as recombinant or transgenic algae. Such algae may be grown in culture or otherwise produced by growing transgenic algal plants. Algae may also be produced recombinantly by methods well known in the art for the purpose of increasing the type of raw materials, such as lipids, especially oils, and hydrocarbons, contemplated for use in the methods of the invention.

In such recombinant methods, host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

By way of non-limiting example, algae cells may be transformed with polynucleotides encoding enzymes that greatly increase the amount of oils and other lipids produced by these cells. For example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The DNA sequence in the expression vector is operatively linked to an appropriate expression control sequence(s) (promoter) to direct mRNA synthesis. As representative examples of such promoters, there may be mentioned: LTR or SV40 promoter, the E. coli, lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transform an appropriate algal host cell to permit the host to express the desired enzyme, thereby greatly increasing the amount of lipids produced by the host. Any of the algal species mentioned herein may be appropriately transformed and used as the host cell.

Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference.

Products obtained from the treating of the invention, such as an oil or lipid, or a carbohydrate, especially a polysaccharide, may be further purified in order to remove any remaining contaminants, thereby producing a purified oil or carbohydrate, which is substantially free of contaminants present in the crude extract. The purified oil is rich in lipids, such as triglycerides, and can be used as a food oil, lubricant, burned directly, or subjected to processing to convert it into a fuel, such as bio-diesel or bio-gasoline. A carbohydrate produced from the algae following solubilization may find use as a pharmaceutical or neutraceutical. The purified oil is suitable for transesterification for use as bio-diesel or bio-gasoline.

The present invention provides processes and compositions for separating a crude extract containing lipids from biological material. The present invention is suitable for extraction of triglycerides for ultimate use as fuel oils, which can be burned directly, or processed further to make fuels such as bio-diesel or bio-gasoline.

Oils produced by the methods herein can be used as a fuel, either directly if fed to a burner or an engine, or indirectly if converted to biodiesel via transesterification. Vegetable oils, derived from plants like soy, canola, sunflower, marigold and palm, can also used as renewable energy resources, usually upon their conversion into biodiesel via transesterification. Oil produced from microorganisms, such as algae, can be used in addition to or as a replacement of said vegetable oils.

One of the desired products from the materials solubilized by the methods of the invention are carbohydrates, such as polysaccharides. The cells of algae are encapsulated within a sulfated polysaccharide, the external part of which can be obtained from the solubilized material produced by the methods of the invention. This solubilized or liquefied product can be extracted to obtain the polysaccharide portion, which can be subsequently purified.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

In carrying out the procedures of the present invention it is to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

What is claimed:

1. A method of treating algae or algal material, comprising treating the said material with a liquid containing at least one member selected from the group consisting of thioacid esters of phosphorus and oxoacid esters of phosphorus, to liquefy at least a portion of said material.

2. The method of claim 1, wherein the liquid contains an oxoacid ester of phosphorus.

3. The method of claim 1, wherein the algae or algal material is fully solubilized as a result of said treating.

4. The method of claim 1, wherein the algae or algal material is macroalgae in its various forms.

5. The method of claim 1, wherein said treating is carried out at a temperature of 20 to 150° C.

6. The method of claim 1, wherein said treating is carried out at a temperature of 80 to 100° C.

7. The method of claim 1, wherein said treating is carried out at a pH range of 1 to 9.

8. The method of claim 2, wherein the oxoacid ester of phosphorus is selected from the group consisting of esters of phosphorous acid, phosphoric acid, hypophosphorous acid, polyphosphoric acid, and mixtures thereof.

9. The method of claim 2, wherein the alcohol portion of the ester is selected from the group consisting of methanol, ethanol, ethylene glycol, propylene glycol, glycerol, pentaerythritol, trimethylol ethane, trimethylol propane, trimethylol alkane, alkanol, polyol, and mixtures thereof.

10. The method of claim 1 further comprising: regulating the water content of the blend before or during said treating.

11. The method of claim 10, wherein said regulating the water content comprises removing water.

12. The method of claim 11, wherein said removing water comprises molecular sieving.

13. The method of claim 11, wherein said removing water comprises distillation.

14. The method of claim 11, wherein said removing water comprises adding a dehydrating agent to the blend.

15. The method of claim 11, wherein said removing water comprises foaming or misting the blend.

16. The method of claim 1 further comprising: sonicating during or after said treating.

17. The method of claim 1 further comprising: adding a bioconversion agent after said treating to bioconvert at least a portion of the liquefied material.

18. The method of claim 17, wherein the bioconversion agent is a methanogen.

19. The method of claim 17, wherein the bioconversion agent is any of a variety of facultative anaerobes, acetogens, and other microbial species.

20. The treated material of the method of claim 1.

21. The bioconverted, treated material of the method of claim 17.

22. A composition comprising solubilized organophosphorous ester derivatives of algae or algal material.

23. The method of claim 18, wherein said treating results in formation of methane.

24. The method of claim 17, wherein said treating results in the formation of hydrocarbons, fatty acids and other useful chemicals and gases.

25. The method of claim 1, further comprising separating the oil from water and non-oil materials in the treated material.
